(19) United States
(12) Patent Application Publication
(10) Pub. No.: US 2024/0156531 A1
(43) Pub. Date: May 16, 2024

(54) METHOD FOR CREATING A SURGICAL PLAN BASED ON AN ULTRASOUND VIEW

(71) Applicant: Medtronic Navigation, Inc., Louisville, CO (US)

(72) Inventors: Tyler S. Stevenson, Westminster, CO (US); Sarah G. LaCoste, Superior, CO (US); Morgan Suzanne Arvisais, Longmont, CO (US); Benjamin Kevin Hendricks, Phoenix, AZ (US)

(21) Appl. No.: 17/988,162

(22) Filed: Nov. 16, 2022

(51) Int. Cl. A61B 34/10; A61B 90/00

(52) U.S. Cl. CPC A61B 34/10; A61B 90/37; A61B 2034/101; A61B 2034/107; A61B 2034/2055

(57) ABSTRACT

Systems and methods are described that generate a first virtual space corresponding to a first multi-dimensional image set, where the first multi-dimensional image set includes one or more images generated based on receiving first imaging signals. The systems and method store pose information of one or more markers in association with a second virtual space, where the second virtual space corresponds to a second multi-dimensional image set including one or more ultrasound images generated based on receiving second imaging signals. The systems and method generate an image-based surgical plan in association with the first virtual space based on the pose information of the one or more markers. In some cases, the systems and method translate the pose information of the one or more markers to the first virtual space, and generating the image-based surgical plan is based on the translation of the pose information to the first virtual space.

### FIELD OF INVENTION

The present disclosure is generally directed to imaging guidance in association with a surgical procedure, and relates more particularly to creating a surgical plan based on an ultrasound image.

### BACKGROUND

Surgical robots may assist a surgeon or other medical provider in carrying out a surgical procedure, or may complete one or more surgical procedures autonomously. Imaging may be used by a medical provider for visual guidance in association with diagnostic and/or therapeutic procedures.

### BRIEF SUMMARY

Example aspects of the present disclosure include:

A system including: a processor; and a memory storing instructions thereon that, when executed by the processor, cause the processor to: generate a first virtual space corresponding to a first multi-dimensional image set; store pose information of one or more markers in association with a second virtual space, wherein the second virtual space corresponds to a second multi-dimensional image set including one or more ultrasound images; and generate an image-based surgical plan in association with the first virtual space based on the pose information of the one or more markers in association with the second virtual space.

Any of the aspects herein, wherein the instructions are further executable by the processor to: translate the pose information of the one or more markers to the first virtual space, wherein generating the image-based surgical plan is based on translating the pose information to the first virtual space.

Any of the aspects herein, further including generating the second virtual space, wherein: generating the second virtual space includes segmenting a third virtual space corresponding to the second multi-dimensional image set; the second virtual space includes a two-dimensional virtual space or a three-dimensional virtual space; and the third virtual space includes a three-dimensional virtual space.

Any of the aspects herein, wherein the instructions are further executable by the processor to: receive an indication of candidate coordinates associated with the image-based surgical plan and the first virtual space; and output, in response to receiving the indication of the candidate coordinates, guidance information associated with at least the image-based surgical plan.

Any of the aspects herein, wherein: the guidance information includes the pose information of the one or more markers in association with the second virtual space; and the pose information of the one or more markers corresponds to the candidate coordinates in the first virtual space.

Any of the aspects herein, wherein the guidance information includes an indication of a target point in the second virtual space, wherein the target point is associated with the one or more markers.

Any of the aspects herein, wherein the guidance information includes an indication of at least one of: a target pose of an image sensor device with respect to a target point in the second virtual space; a target trajectory of the image sensor device with respect to the target point in the second virtual space; and a hind point of the target trajectory.

Any of the aspects herein, wherein the guidance information includes an indication of at least one of: a target pose of an image sensor device with respect to a target point in a physical space corresponding to the second virtual space; a target trajectory of the image sensor device with respect to the target point in the physical space; and a hind point of the target trajectory.

Any of the aspects herein, wherein: the guidance information includes alignment information associated with current pose information of an image sensor device and stored pose information of the image sensor device; and the stored pose information of the image sensor device correlates to the candidate coordinates associated with the image-based surgical plan and the first virtual space.

Any of the aspects herein, wherein the instructions are further executable by the processor to adjust one or more settings associated with an image sensor device based on the guidance information.

Any of the aspects herein, wherein the instructions are further executable by the processor to at least one of: deliver therapy to a subject based on at least one of the one or more markers and the image-based surgical plan, wherein delivering the therapy includes transmitting one or more therapeutic ultrasound signals toward a region associated with the one or more markers; and deliver diagnostics data associated with the subject based on at least one of the one or more markers and the image-based surgical plan.

Any of the aspects herein, wherein the one or more markers correspond to one or more anatomical elements included in the one or more ultrasound images.

Any of the aspects herein, wherein generating the image-based surgical plan includes mapping one or more parameters of a surgical task included in the image-based surgical plan to the pose information of the one or more markers.

Any of the aspects herein, wherein storing the pose information of the one or more markers is in response to a trigger condition.

Any of the aspects herein, wherein the instructions are further executable by the processor to: transmit one or more ultrasound signals in a physical space corresponding to the second virtual space; and capture the one or more ultrasound images based on the one or more ultrasound signals.

Any of the aspects herein, wherein: wherein the first multi-dimensional image set includes one or more magnetic resonance imaging (MM) images, one or more computed tomography (CT) images, or one or more multi-dimensional fluoroscopic images.

Any of the aspects herein, wherein: the first multi-dimensional image set includes one or more preoperative images, one or more first intraoperative images, or both; and the second multi-dimensional image set includes one or more second preoperative images, one or more second intraoperative images, or both.

A system including: an interface to receive one or more imaging signals; a processor; and a memory storing data thereon that, when processed by the processor, cause the processor to: generate a first virtual space corresponding to a first multi-dimensional image set, wherein the first multi-dimensional image set includes one or more images generated based on first imaging signals; store pose information of one or more markers in association with a second virtual space, wherein the second virtual space corresponds to a second multi-dimensional image set including one or more ultrasound images generated based on second imaging signals; and generate an image-based surgical plan in association with the first virtual space based on the pose information of the one or more markers.

Any of the aspects herein, wherein the instructions are further executable by the processor to: translate the pose information of the one or more markers to the first virtual space, wherein generating the image-based surgical plan is based on translating the pose information to the first virtual space.

A method including: generating a first virtual space corresponding to a first multi-dimensional image set, wherein the first multi-dimensional image set includes one or more images generated based on receiving first imaging signals; storing pose information of one or more markers in association with a second virtual space, wherein the second virtual space corresponds to a second multi-dimensional image set including one or more ultrasound images generated based on receiving second imaging signals; and generating an image-based surgical plan in association with the first virtual space based on the pose information of the one or more markers.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/implementations in combination with any one or more other aspects/features/implementations.

Use of any one or more of the aspects or features as disclosed herein.

It is to be appreciated that any feature described herein can be claimed in combination with any other feature(s) as described herein, regardless of whether the features come from the same described implementation.

The details of one or more aspects of the disclosure are set forth in the accompanying drawings and the description below. Other features, objects, and advantages of the techniques described in this disclosure will be apparent from the description and drawings, and from the claims.

The preceding is a simplified summary of the disclosure to provide an understanding of some aspects of the disclosure. This summary is neither an extensive nor exhaustive overview of the disclosure and its various aspects, implementations, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, implementations, and configurations of the disclosure are possible utilizing, alone or in combination, one or more of the features set forth above or described in detail below.

Numerous additional features and advantages of the present disclosure will become apparent to those skilled in the art upon consideration of the implementation descriptions provided hereinbelow.

### BRIEF DESCRIPTION OF THE SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, implementations, and configurations of the disclosure, as illustrated by the drawings referenced below.

FIGS. 1A and 1B illustrate examples of a system in accordance with aspects of the present disclosure.

FIGS. 2A and 2B are diagrams illustrating aspects of generating a virtual multi-dimensional space in accordance with aspects of the present disclosure.

FIG. 3 illustrates an example of a process flow in accordance with aspects of the present disclosure.

FIG. 4 illustrates an example of a process flow in accordance with aspects of the present disclosure.

### DETAILED DESCRIPTION

It should be understood that various aspects disclosed herein may be combined in different combinations than the combinations specifically presented in the description and accompanying drawings. It should also be understood that, depending on the example or implementation, certain acts or events of any of the processes or methods described herein may be performed in a different sequence, and/or may be added, merged, or left out altogether (e.g., all described acts or events may not be necessary to carry out the disclosed techniques according to different implementations of the present disclosure). In addition, while certain aspects of this disclosure are described as being performed by a single module or unit for purposes of clarity, it should be understood that the techniques of this disclosure may be performed by a combination of units or modules associated with, for example, a computing device and/or a medical device.

In one or more examples, the described methods, processes, and techniques may be implemented in hardware, software, firmware, or any combination thereof. If implemented in software, the functions may be stored as one or more instructions or code on a computer-readable medium and executed by a hardware-based processing unit. Alternatively or additionally, functions may be implemented using machine learning models, neural networks, artificial neural networks, or combinations thereof (alone or in combination with instructions). Alternatively or additionally, functions may be implemented using machine learning models, neural networks, artificial neural networks, or combinations thereof (alone or in combination with instructions). Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors (e.g., Intel Core i3, i5, i7, or i9 processors; Intel Celeron processors; Intel Xeon processors; Intel Pentium processors; AMD Ryzen processors; AMD Athlon processors; AMD Phenom processors; Apple A10 or 10X Fusion processors; Apple A11, A12, A12X, A12Z, or A13 Bionic processors; or any other general purpose microprocessors), graphics processing units (e.g., Nvidia GeForce RTX 2000-series processors, Nvidia GeForce RTX 3000-series processors, AMD Radeon RX 5000-series processors, AMD Radeon RX 6000-series processors, or any other graphics processing units), application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

Before any implementations of the disclosure are explained in detail, it is to be understood that the disclosure is not limited in its application to the details of construction and the arrangement of components set forth in the following description or illustrated in the drawings. The disclosure is capable of other implementations and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

The terms proximal and distal are used in this disclosure with their conventional medical meanings, proximal being closer to the operator or user of the system, and further from the region of surgical interest in or on the patient, and distal being closer to the region of surgical interest in or on the patient, and further from the operator or user of the system.

In some navigation systems, when navigating an ultrasound image, a user may view a portion of an anatomical element of a patient (e.g., a current slice of the brain) that corresponds to the location of an ultrasound probe. Such navigations systems, however, fail to provide a mechanism for the user to easily return to viewing a previous position.

Aspects of the present disclosure support one or more surgical software features that allow a user to easily return to a particular location the user identified with an ultrasound probe. For example, a system described herein may create either a surgical plan (or an empty ultrasound fan) and lock the plan (or ultrasound fan) into place. Accordingly, for example, the system may provide an improved mechanism via which a user may capture a particular plane or location within an anatomical element (e.g., brain, spine, etc.) using an ultrasound probe. Aspects of the present disclosure support returning to the captured plane or location with the ultrasound probe.

In some aspects, techniques described herein include trajectory planning that includes stereotactic placement of ultrasound guided markers for translation between different virtual spaces. For example, the techniques described herein support translation between an imaging space (e.g., magnetic resonance imaging (MM) space, computed tomography (CT) space, fluoroscopy space, etc.) and another imaging space (e.g., an ultrasound space). Some aspects of the trajectory planning include focal point recall with robotics to fixate a focal point of an instrument (e.g., an ultrasound probe, a microscope, etc.) on an ultrasound guided marker generated in the ultrasound space. Example aspects of a system described herein support generating or storing a marker made in an ultrasound virtual space and translating information (e.g., pose information, etc.) associated with the marker to an MM virtual space.

The term “pose information” used herein may include position (e.g., coordinates), orientation, and trajectory of an object (e.g., a physical object, a virtual object, an imaging device, etc.) with respect to a reference coordinate system. In some cases, the “pose information” may include a relative position, orientation, and trajectory of the object with respect to another object.

Aspects of the present disclosure support implementations using a robotic system. For example, the robotic system may establish virtual spaces (e.g., ultrasound space, MM space, etc.) and map tasks to the virtual spaces using the techniques described herein. The robotic system may support storing target points (e.g., focal points) and probe positional information with respect to one virtual space (e.g., ultrasound space) autonomously and/or semi-autonomously based on a user input. In some examples, the probe positional information may include position, orientation, trajectory, and the like with respect to an X-axis, a Y-axis, and/or a Z-axis. The robotic system may support translating the target points and probe positional information from the virtual space to another virtual space (e.g., MM space, CT space, fluoroscopy space, etc.). In some example implementations, the robotic system may support recalling stored target points and probe positional information.

Implementations of the present disclosure provide technical solutions to one or more problems of user unfamiliarity with an ultrasound space. For example, the techniques described herein for translating virtual ultrasound markers to virtual markers in a virtual space (e.g., MRI space, CT space, etc.) a surgeon is familiar with may provide improved convenience and increased accessibility to data associated with surgical procedures.

Other implementations of the present disclosure provide a mechanism for a surgeon to easily return to viewing a previous position in the ultrasound space. For example, the surgeon may need to pause a medical procedure (e.g., examining an anatomical element using an ultrasound sensor, delivering therapy using the ultrasound sensor, performing a surgical operation while viewing an anatomical element using the ultrasound sensor, etc.). The techniques described herein support saving the pose information and settings of the ultrasound sensor, thereby providing a mechanism which reduces the amount of time associated with returning to a paused medical procedure.

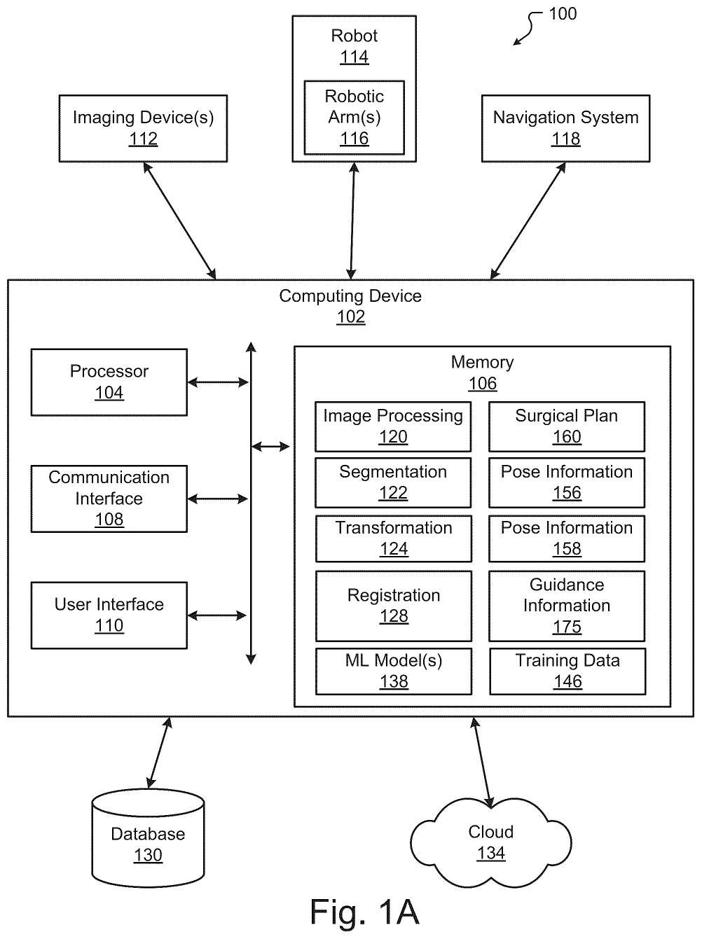

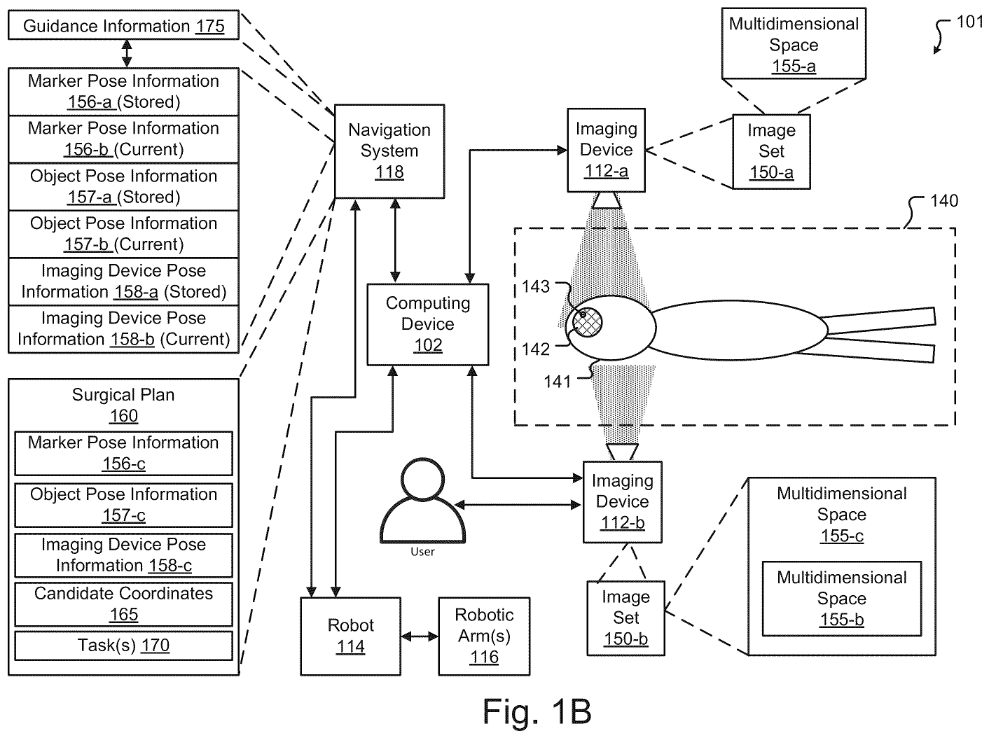

FIG. 1A illustrates an example of a system 100 that supports aspects of the present disclosure.

The system 100 includes a computing device 102, one or more imaging devices 112, a robot 114, a navigation system 118, a database 130, and/or a cloud network 134 (or other network). Systems according to other implementations of the present disclosure may include more or fewer components than the system 100. For example, the system 100 may omit and/or include additional instances of one or more components of the computing device 102, the imaging device(s) 112, the robot 114, navigation system 118, the database 130, and/or the cloud network 134. In an example, the system 100 may omit any instance of the computing device 102, the imaging device(s) 112, the robot 114, navigation system 118, the database 130, and/or the cloud network 134. For example, the system 100 may omit the robot 114 and the navigation system 118. The system 100 may support the implementation of one or more other aspects of one or more of the methods disclosed herein.

The computing device 102 includes a processor 104, a memory 106, a communication interface 108, and a user interface 110. Computing devices according to other implementations of the present disclosure may include more or fewer components than the computing device 102.

The processor 104 of the computing device 102 may be any processor described herein or any similar processor. The processor 104 may be configured to execute instructions stored in the memory 106, which instructions may cause the processor 104 to carry out one or more computing steps utilizing or based on data received from the imaging devices 112, the robot 114, the navigation system 118, the database 130, and/or the cloud network 134.

The memory 106 may be or include RAM, DRAM, SDRAM, other solid-state memory, any memory described herein, or any other tangible, non-transitory memory for storing computer-readable data and/or instructions. The memory 106 may store information or data associated with completing, for example, any step of the methods (e.g., process flow 300, process flow 400) described herein, or of any other methods. The memory 106 may store, for example, instructions and/or machine learning models that support one or more functions of the imaging devices 112, the robot 114, and the navigation system 118. For instance, the memory 106 may store content (e.g., instructions and/or machine learning models) that, when executed by the processor 104, enable image processing 120, segmentation 122, transformation 124, and/or registration 128. The memory 106 may store content one or more surgical plans 160, pose information (e.g., pose information 156, pose information 157, pose information 158), and guidance information 175, example aspects of which are later described with reference to FIG. 1B. Such content, if provided as in instruction, may, in some implementations, be organized into one or more applications, modules, packages, layers, or engines.

Alternatively or additionally, the memory 106 may store other types of content or data (e.g., machine learning models, artificial neural networks, deep neural networks, etc.) that can be processed by the processor 104 to carry out the various method and features described herein. Thus, although various contents of memory 106 may be described as instructions, it should be appreciated that functionality described herein can be achieved through use of instructions, algorithms, and/or machine learning models. The data, algorithms, and/or instructions may cause the processor 104 to manipulate data stored in the memory 106 and/or received from or via the imaging devices 112, the robot 114, the navigation system 118, the database 130, and/or the cloud network 134.

The computing device 102 may also include a communication interface 108. The communication interface 108 may be used for receiving data or other information from an external source (e.g., the imaging devices 112, the robot 114, the navigation system 118, the database 130, the cloud network 134, and/or any other system or component separate from the system 100), and/or for transmitting instructions, data (e.g., image data, stored surgical plans, guidance information, pose information, measurements, temperature information, etc.), or other information to an external system or device (e.g., another computing device 102, the imaging devices 112, the robot 114, the navigation system 118, the database 130, the cloud network 134, and/or any other system or component not part of the system 100). The communication interface 108 may include one or more wired interfaces (e.g., a USB port, an Ethernet port, a Firewire port) and/or one or more wireless transceivers or interfaces (configured, for example, to transmit and/or receive information via one or more wireless communication protocols such as 802.11a/b/g/n, Bluetooth, NFC, ZigBee, and so forth). In some implementations, the communication interface 108 may support communication between the device 102 and one or more other processors 104 or computing devices 102, whether to reduce the time needed to accomplish a computing-intensive task or for any other reason.

The computing device 102 may also include one or more user interfaces 110. The user interface 110 may be or include a keyboard, mouse, trackball, monitor, television, screen, touchscreen, and/or any other device for receiving information from a user and/or for providing information to a user. The user interface 110 may be used, for example, to receive a user selection or other user input regarding any step of any method described herein. Notwithstanding the foregoing, any required input for any step of any method described herein may be generated automatically by the system 100 (e.g., by the processor 104 or another component of the system 100) or received by the system 100 from a source external to the system 100. In some implementations, the user interface 110 may support user modification (e.g., by a surgeon, medical personnel, a patient, etc.) of instructions to be executed by the processor 104 according to one or more implementations of the present disclosure, and/or to user modification or adjustment of a setting of other information displayed on the user interface 110 or corresponding thereto.

In some implementations, the computing device 102 may utilize a user interface 110 that is housed separately from one or more remaining components of the computing device 102. In some implementations, the user interface 110 may be located proximate one or more other components of the computing device 102, while in other implementations, the user interface 110 may be located remotely from one or more other components of the computer device 102.

The imaging device 112 may be operable to image anatomical feature(s) (e.g., a bone, veins, tissue, vascular structures, etc.) and/or other aspects of patient anatomy to yield image data (e.g., image data depicting or corresponding to a bone, veins, tissue, etc.). “Image data” as used herein refers to the data generated or captured by an imaging device 112, including in a machine-readable form, a graphical/visual form, and in any other form. In various examples, the image data may include data corresponding to an anatomical feature of a patient, or to a portion thereof. The image data may be or include a preoperative image, an intraoperative image, a postoperative image, or an image taken independently of any surgical procedure. In some implementations, a first imaging device 112 may be used to obtain first image data (e.g., a first image) at a first time, and a second imaging device 112 may be used to obtain second image data (e.g., a second image) at a second time after the first time.

The imaging device 112 may be capable of taking a 2D image or a 3D image to yield the image data. The imaging device 112 may be or include, for example, an ultrasound scanner (which may include, for example, a physically separate transducer and receiver, or a single ultrasound transceiver), an O-arm, a C-arm, a G-arm, or any other device utilizing X-ray-based imaging (e.g., a fluoroscope, a CT scanner, or other X-ray machine), a magnetic resonance imaging (MM) scanner, an optical coherence tomography (OCT) scanner, an endoscope, a microscope, an optical camera, a thermographic camera (e.g., an infrared camera), a radar system (which may include, for example, a transmitter, a receiver, a processor, and one or more antennae), or any other imaging device 112 suitable for obtaining images of an anatomical feature of a patient. In the example case of an ultrasound scanner, the imaging device 112 may support doppler ultrasound. The imaging device 112 may be contained entirely within a single housing, or may include a transmitter/emitter and a receiver/detector that are in separate housings or are otherwise physically separated.

In some implementations, the imaging device 112 may include more than one imaging device 112. For example, a first imaging device may provide first image data and/or a first image, and a second imaging device may provide second image data and/or a second image. In still other implementations, the same imaging device may be used to provide both the first image data and the second image data, and/or any other image data described herein. The imaging device 112 may be operable to generate a stream of image data. For example, the imaging device 112 may be configured to operate with an open shutter, or with a shutter that continuously alternates between open and shut so as to capture successive images. For purposes of the present disclosure, unless specified otherwise, image data may be considered to be continuous and/or provided as an image data stream if the image data represents two or more frames per second.

The robot 114 may be any surgical robot or surgical robotic system. The robot 114 may be or include, for example, the Mazor X™ Stealth Edition robotic guidance system. The robot 114 may be configured to position the imaging device 112 at one or more precise position(s) and orientation(s), and/or to return the imaging device 112 to the same position(s) and orientation(s) at a later point in time. The robot 114 may additionally or alternatively be configured to manipulate a surgical tool (whether based on guidance from the navigation system 118 or not) to accomplish or to assist with a surgical task. In some implementations, the robot 114 may be configured to hold and/or manipulate an anatomical element during or in connection with a surgical procedure. The robot 114 may include one or more robotic arms 116. In some implementations, the robotic arm 116 may include a first robotic arm and a second robotic arm, though the robot 114 may include more than two robotic arms. In some implementations, one or more of the robotic arms 116 may be used to hold and/or maneuver the imaging device 112. In implementations where the imaging device 112 includes two or more physically separate components (e.g., a transmitter and receiver), one robotic arm 116 may hold one such component, and another robotic arm 116 may hold another such component. Each robotic arm 116 may be positionable independently of the other robotic arm. The robotic arms 116 may be controlled in a single, shared coordinate space, or in separate coordinate spaces.

The robot 114, together with the robotic arm 116, may have, for example, one, two, three, four, five, six, seven, or more degrees of freedom. Further, the robotic arm 116 may be positioned or positionable in any pose, plane, and/or focal point. The pose includes a position and an orientation. As a result, an imaging device 112, surgical tool, or other object held by the robot 114 (or, more specifically, by the robotic arm 116) may be precisely positionable in one or more needed and specific positions and orientations.

The robotic arm(s) 116 may include one or more sensors that enable the processor 104 (or a processor of the robot 114) to determine a precise pose in space of the robotic arm (as well as any object or element held by or secured to the robotic arm).

In some implementations, reference markers (e.g., navigation markers) may be placed on the robot 114 (including, e.g., on the robotic arm 116), the imaging device 112, or any other object in the surgical space. The reference markers may be tracked by the navigation system 118, and the results of the tracking may be used by the robot 114 and/or by an operator of the system 100 or any component thereof. In some implementations, the navigation system 118 can be used to track other components of the system (e.g., imaging device 112) and the system can operate without the use of the robot 114 (e.g., with the surgeon manually manipulating the imaging device 112 and/or one or more surgical tools, based on information and/or instructions generated by the navigation system 118, for example).

The navigation system 118 may provide navigation for a surgeon and/or a surgical robot during an operation. The navigation system 118 may be any now-known or future-developed navigation system, including, for example, the Medtronic StealthStation™ S8 surgical navigation system or any successor thereof. The navigation system 118 may include one or more cameras or other sensor(s) for tracking one or more reference markers, navigated trackers, or other objects within the operating room or other room in which some or all of the system 100 is located. The one or more cameras may be optical cameras, infrared cameras, or other cameras. In some implementations, the navigation system 118 may include one or more electromagnetic sensors. In various implementations, the navigation system 118 may be used to track a position and orientation (e.g., a pose) of the imaging device 112, the robot 114 and/or robotic arm 116, and/or one or more surgical tools (or, more particularly, to track a pose of a navigated tracker attached, directly or indirectly, in fixed relation to the one or more of the foregoing). The navigation system 118 may include a display for displaying one or more images from an external source (e.g., the computing device 102, imaging device 112, or other source) or for displaying an image and/or video stream from the one or more cameras or other sensors of the navigation system 118. In some implementations, the system 100 can operate without the use of the navigation system 118. The navigation system 118 may be configured to provide guidance (e.g., guidance information 175) to a surgeon or other user of the system 100 or a component thereof, to the robot 114, or to any other element of the system 100 regarding, for example, a pose of one or more anatomical elements, whether or not a tool is in the proper trajectory, and/or how to move a tool into the proper trajectory to carry out a surgical task according to a preoperative or other surgical plan (e.g., a surgical plan 160).

The processor 104 may utilize data stored in memory 106 as a neural network. The neural network may include a machine learning architecture. In some aspects, the neural network may be or include one or more classifiers. In some other aspects, the neural network may be or include any machine learning network such as, for example, a deep learning network, a convolutional neural network, a reconstructive neural network, a generative adversarial neural network, or any other neural network capable of accomplishing functions of the computing device 102 described herein. Some elements stored in memory 106 may be described as or referred to as instructions or instruction sets, and some functions of the computing device 102 may be implemented using machine learning techniques.

For example, the processor 104 may support machine learning model(s) 138 which may be trained and/or updated based on data (e.g., training data 146) provided or accessed by any of the computing device 102, the imaging device 112, the robot 114, the navigation system 118, the database 130, and/or the cloud network 134. The machine learning model(s) 138 may be built and updated by the monitoring engine 126 based on the training data 146 (also referred to herein as training data and feedback).

The database 130 may store information that correlates one coordinate system to another (e.g., one or more robotic coordinate systems to a patient coordinate system and/or to a navigation coordinate system). The database 130 may additionally or alternatively store, for example, one or more surgical plans (including, for example, pose information about a target and/or image information about a patient's anatomy at and/or proximate the surgical site, for use by the robot 114, the navigation system 118, and/or a user of the computing device 102 or of the system 100); one or more images useful in connection with a surgery to be completed by or with the assistance of one or more other components of the system 100; and/or any other useful information.

The database 130 may be configured to provide any such information to the computing device 102 or to any other device of the system 100 or external to the system 100, whether directly or via the cloud network 134. In some implementations, the database 130 may be or include part of a hospital image storage system, such as a picture archiving and communication system (PACS), a health information system (HIS), and/or another system for collecting, storing, managing, and/or transmitting electronic medical records including image data.

In some aspects, the computing device 102 may communicate with a server(s) and/or a database (e.g., database 130) directly or indirectly over a communications network (e.g., the cloud network 134). The communications network may include any type of known communication medium or collection of communication media and may use any type of protocols to transport data between endpoints. The communications network may include wired communications technologies, wireless communications technologies, or any combination thereof.

Wired communications technologies may include, for example, Ethernet-based wired local area network (LAN) connections using physical transmission mediums (e.g., coaxial cable, copper cable/wire, fiber-optic cable, etc.). Wireless communications technologies may include, for example, cellular or cellular data connections and protocols (e.g., digital cellular, personal communications service (PCS), cellular digital packet data (CDPD), general packet radio service (GPRS), enhanced data rates for global system for mobile communications (GSM) evolution (EDGE), code division multiple access (CDMA), single-carrier radio transmission technology (1×RTT), evolution-data optimized (EVDO), high speed packet access (HSPA), universal mobile telecommunications service (UMTS), 3G, long term evolution (LTE), 4G, and/or 5G, etc.), Bluetooth®, Bluetooth® low energy, Wi-Fi, radio, satellite, infrared connections, and/or ZigBee® communication protocols.

The Internet is an example of the communications network that constitutes an Internet Protocol (IP) network consisting of multiple computers, computing networks, and other communication devices located in multiple locations, and components in the communications network (e.g., computers, computing networks, communication devices) may be connected through one or more telephone systems and other means. Other examples of the communications network may include, without limitation, a standard Plain Old Telephone System (POTS), an Integrated Services Digital Network (ISDN), the Public Switched Telephone Network (PSTN), a Local Area Network (LAN), a Wide Area Network (WAN), a wireless LAN (WLAN), a Session Initiation Protocol (SIP) network, a Voice over Internet Protocol (VoIP) network, a cellular network, and any other type of packet-switched or circuit-switched network known in the art. In some cases, the communications network 120 may include of any combination of networks or network types. In some aspects, the communications network may include any combination of communication mediums such as coaxial cable, copper cable/wire, fiber-optic cable, or antennas for communicating data (e.g., transmitting/receiving data).

The computing device 102 may be connected to the cloud network 134 via the communication interface 108, using a wired connection, a wireless connection, or both. In some implementations, the computing device 102 may communicate with the database 130 and/or an external device (e.g., a computing device) via the cloud network 134.

The system 100 or similar systems may be used, for example, to carry out one or more aspects of any of the methods (e.g., process flow 300, process flow 400, etc.) described herein. The system 100 or similar systems may also be used for other purposes.

FIG. 1B illustrates an example 101 of the system 100 that supports aspects of the present disclosure. Aspects of the example 101 may be implemented by the computing device 102, imaging devices 112, robot 114 (e.g., a robotic system), and navigation system 118. For example, example aspects of FIG. 1B described as being implemented by computing device 102 may be implemented by or in combination with robot 114 and/or navigation system 118.

In the example of FIG. 1B, the imaging device 112-a may be an Mill imaging device and the imaging device 112-b may be an ultrasound imaging device (e.g., an ultrasound sensor). Each of the imaging devices 112 may be capable of capturing image data. For example, each of the imaging devices 112 may be capable of capturing respective multi-dimensional image sets 150 with respect to an environment 140 and/or objects (e.g., a patient 141, an anatomical element 142 of the patient 141, etc.) in the environment 140. The imaging devices 112 and/or computing device 102 may be capable of generating virtual spaces (e.g., multi-dimensional spaces 155) respectively corresponding to the multi-dimensional image sets 150. In some examples, the environment 140 may be a physical space such as an operating room, a hospital room, a laboratory, or the like.

It is to be understood that the imaging devices 112 are not limited thereto, and the examples described with reference to FIG. 1B may be implemented using any imaging device 112 described herein. For example, aspects of the present disclosure support implementations in which the imaging device 112-a is a CT scanner and the image set 150-a includes CT images. In another example implementation the imaging device 112-a is a fluoroscopy scanner and the image set 150-a includes fluoroscopic images.

An example implementation associated with creating a surgical plan 160 based on an ultrasound view is described herein. The computing device 102 may generate a multi-dimensional space 155-a corresponding to an image set 150-a captured by imaging device 112-a. In the example in which the imaging device 112-a is an Mill scanner, the image set 150-a may include Mill images. The computing device 102 may further generate a multi-dimensional space 155-b corresponding to an image set 150-b captured by imaging device 112-b. In the example in which the imaging device 112-b is an ultrasound scanner, the image set 150-b may include ultrasound images.

The imaging device 112-b may transmit ultrasound signals in the environment 140. For example, the imaging device 112-b may transmit ultrasound signals toward the environment 140 (or region thereof), the subject 141, and/or the anatomical element 142. The imaging device 112-b may capture ultrasound images based on a response signal produced by the ultrasound signals. Images in the image set 150-a and image set 150-b may include preoperative images and/or intraoperative images. For example, the computing device 102 may generate and update the multi-dimensional space 155-a and/or the multi-dimensional space 155-b prior to or during a surgical procedure.

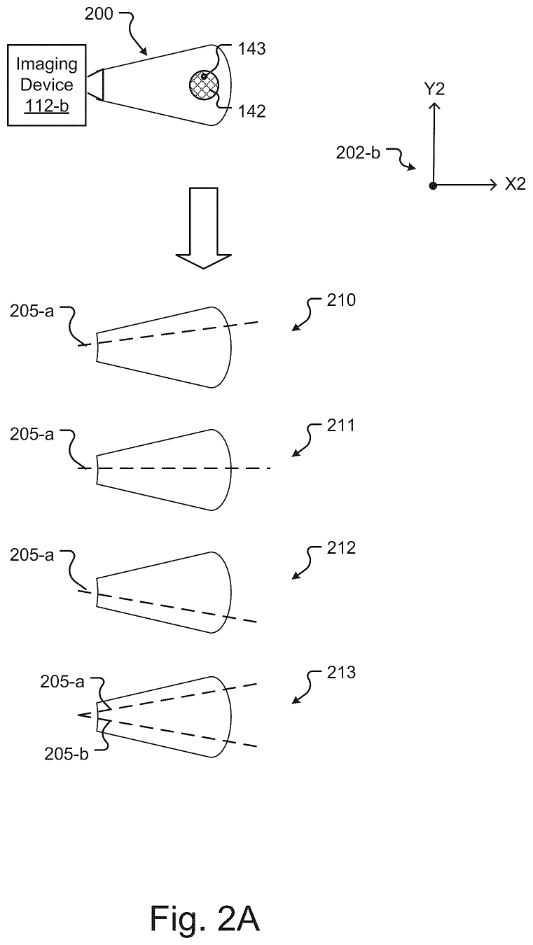

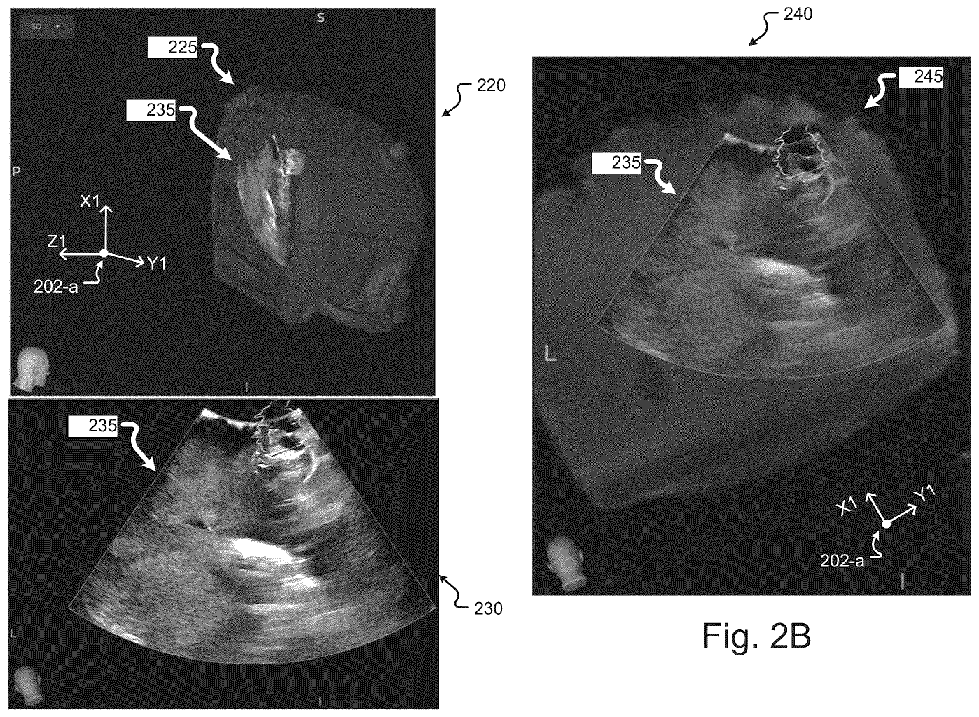

In an example of generating the multi-dimensional space 155-b, the imaging device 112-b or computing device 102 may first generate a multi-dimensional space 155-c corresponding to the image set 150-b. For example, the multi-dimensional space 155-c may be a three-dimensional virtual space that includes or represents the environment 140, the subject 141, and/or the anatomical element 142. The multi-dimensional space 155-c may correspond to an ultrasound fan (e.g., ultrasound fan 200 later described with reference to FIG. 2A) generated by the imaging device 112-b.

The computing device 102 may generate the multi-dimensional space 155-b by segmenting the multi-dimensional space 155-c (e.g., cutting away a portion of the multi-dimensional space 155-c), such that the multi-dimensional space 155-b is a three-dimensional space. In some cases, generating the multi-dimensional space 155-b may include incrementally segmenting the multi-dimensional space 155-c. In some other examples, generating the multi-dimensional space 155-b may include bisecting the multi-dimensional space 155-c, such that the multi-dimensional space 155-b is a two-dimensional space. Example aspects of the multi-dimensional space 155-b and the multi-dimensional space 155-c are later described with reference to FIG. 2A and FIG. 2B.

The computing device 102 may establish a marker 143 (or multiple markers 143) in the multi-dimensional space 155-b. The marker 143 may be, for example, a virtual marker or virtual landmark in the multi-dimensional space 155-b. In some aspects, the marker 143 may correspond to the anatomical element 142 (or a portion thereof). The computing device 102 may establish the marker 143 in response to a trigger condition. Non-limiting examples of the trigger condition include a user input at a user interface (e.g., a touch screen display, etc.) of the computing device 102, a user input (e.g., a button press) at a physical interface of the imaging device 112-b, a user input at a physical interface (e.g., a foot pedal, a set of physical buttons, a remote control, etc.) electrically or wirelessly coupled to the computing device 102 or the imaging device 112-b, and a voice input detected by the computing device 102. In some examples, the trigger condition may include a software algorithm-based trigger. For example, the computing device 102 may predict a region(s) of interest within the image acquisition to be tumor, and the computing device 102 may establish a marker 143 (or multiple markers 143) in the multi-dimensional space 155-b that corresponds to the region(s) of interest. Accordingly, for example, the computing device 102 may tag the region(s) of interest for further review, using the marker 143.

The computing device 102 may store pose information 156-a (also referred to herein as “marker pose information”) of the marker 143 in association with the multi-dimensional space 155-b.

In some aspects, the computing device 102 may store pose information 157-a (also referred to herein as “object pose information”) of the anatomical element 142 and/or subject 141 in association with the multi-dimensional space 155-b.

In some aspects, the computing device 102 may store pose information 158-a (also referred to herein as “imaging device pose information”) of the imaging device 112-b in association with the multi-dimensional space 155-b.

In some cases, the pose information 156-a, the pose information 157-a, and the pose information 158-a may be referred to as “stored pose information.” The computing device 102 may recall the stored pose information in response to a user request, thereby enabling the user to easily return to a location identified by the user with the imaging device 112-b. The stored pose information may enable the user to position the imaging device 112-b back to the same position, orientation, and/or trajectory when the pose information was initially stored.

In an example, the pose information 156-a, the pose information 157-a, and the pose information 158-a may be stored with respect to any of a coordinate system associated with the imaging device 112-b, a coordinate system associated with the environment 140, a coordinate system associated with the subject 141, a coordinate system associated with the multi-dimensional space 155-b, and a coordinate system associated with the multi-dimensional space 155-c. In some aspects, the pose information 158-a of the imaging device 112-b may include position and orientation of the imaging device 112-b with respect to the subject 141, the anatomical element 142, and/or the marker 143. In some examples, the coordinate system of the multi-dimensional space 155-b may be the same as the coordinate system of the imaging device 112-b.

The computing device 102 may store the pose information 156-a, the pose information 157-a and/or the pose information 158-a in response to any example trigger condition described herein. For example, the computing device 102 may store the pose information 156-a, the pose information 157-a, and/or the pose information 158-a in response to a user input at a user interface (e.g., a touch screen display, etc.) of the computing device 102, a user input (e.g., a button press) at the imaging device 112-b, a user input at a physical interface (e.g., a foot pedal, a set of physical buttons, etc.) electrically coupled to the computing device 102 or the imaging device 112-b, and/or a voice input.

The computing device 102 may translate the pose information 156-a, the pose information 157-a, and/or the pose information 158-a of imaging device 112-b to the multi-dimensional space 155-a. For example, the computing device 102 may map or correlate the pose information 156-a to pose information 156-c (also referred to herein as “marker pose information”) according to the multi-dimensional space 155-a. In another example, the computing device 102 may map or correlate the pose information 157-a to pose information 157-c (also referred to herein as “object pose information”) according to the multi-dimensional space 155-a. In another example, the computing device 102 may map or correlate the pose information 158-a to pose information 158-c (also referred to herein as “imaging device pose information”) according to the multi-dimensional space 155-a. The pose information 156-c, the pose information 157-c, and the pose information 158-c may be with respect to a coordinate system of the multi-dimensional space 155-a.

The computing device 102 may generate a surgical plan 160 (e.g., an image-based surgical plan) in association with the multi-dimensional space 155-a based on the translation of the pose information 156-a, the pose information 157-b, and/or the pose information 158-a to the multi-dimensional space 155-a. For example, the surgical plan 160 may include the pose information 156-c, the pose information 157-c, and the pose information 158-c.

In some aspects, the computing device 102 may map a task(s) 170 (e.g., surgical task(s), surgical actions, etc.) included in the surgical plan 160 to the pose information 156-c, the pose information 157-c, and/or the pose information 158-c expressed in relation to the multi-dimensional space 155-a. Accordingly, for example, the system 100 may enable a user (e.g., an operator, a surgeon, etc.) to view any task(s) 170 with respect to the multi-dimensional space 155-a with which the user is more familiar or experienced. In some aspects, the task(s) 170 may include one or more operations which leverage the robot 114 for either diagnostic or therapeutic purposes. In an example, the task(s) 170 may include a diagnostic evaluation of the subject 141 to confirm or exclude a suspected medical condition, assess the efficacy of a treatment plan (e.g., through repeated monitoring), assess a treatment outcome or progression of a medical condition, perform a medical screening, and the like. In some aspects, the computing device 102 may generate and output diagnostics data based on a task(s) 170 such as a diagnostic evaluation.

An example implementation associated with recalling a marker(s) 143 and/or a task(s) 170 via the surgical plan 160 is described herein. In some aspects, the example implementation supports recalling saved pose information (e.g., pose information 156-a through pose information 158-a) in the multi-dimensional space 155-b.

The computing device 102 may receive an indication of candidate coordinates 165 in the multi-dimensional space 155-a. In some cases, the candidate coordinates 165 may be associated with a region in the multi-dimensional space 155-a. Additionally, or alternatively, the computing device 102 may receive an indication of a stored task(s) 170 that the user wishes to perform. In an example, the stored task 170 may be a partially completed task which the user wishes to complete.

In some aspects, the user may provide an input selecting the candidate coordinates 165 and/or the stored task 170 via the user interface 110 of the computing device 102. For example, the computing device 102 may display the surgical plan 160, stored tasks 170 selectable in association with the surgical plan 170, and one or more sets of candidate coordinates 165 selectable in association with the surgical plan 170.

In response to receiving the indication of the candidate coordinates 165 and/or a stored task(s) 170, the computing device 102 may output guidance information 175. The guidance information 175 may include the pose information 156-a (stored pose information) of the marker 143 and/or the pose information 157-a of the anatomical element 142 in association with the multi-dimensional space 155-b. In some aspects, the pose information 156-a and/or the pose information 157-a in the multi-dimensional space 155-b are correlated to the candidate coordinates 165 in the multi-dimensional space 155-a.

The guidance information 175 may include an indication of a target point in the multi-dimensional space 155-b. In some aspects, the target point may be a target focal point associated with examining the subject 141 and/or delivering treatment to the subject 141 with the imaging device 112-b. In some aspects, the target point may correspond to coordinates of the marker 143 or the anatomical element 142 in the multi-dimensional space 155-b.

In some other aspects, the guidance information 175 may include an indication of a target pose (e.g., coordinates and/or orientation) of the imaging device 112-b with respect to the target point. In some cases, the guidance information 175 may include an indication of a target trajectory of the imaging device 112-b with respect to the target point. Accordingly, for example, the guidance information 175 may indicate how to position the imaging device 112-b in association with examining and/or delivering therapy to an anatomical element 142 located at the target point. In some aspects, the target pose and the target trajectory may be a pose and trajectory previously stored in response to a user input as described herein. The target pose and the target trajectory of the imaging device 112-b may be stored in the pose information 158-a. In some aspects, the guidance information 175 may include an indication of a hind point (along with the target point) of a target trajectory. For example, the computing device 102 may register the hind point along with the target point to provide the target trajectory.

In some example aspects, the guidance information 175 may include alignment information associated with current pose information 158-b of the imaging device 112-b and the pose information 158-a (i.e., stored pose information) of the imaging device 112-b. For example, to facilitate returning the imaging device 112-b to the stored pose, the guidance information 175 indicates whether the actual pose of the imaging device 112-b is aligned with the stored pose. In an example, the guidance information 175 may indicate how to position the imaging device 112-b in association with aligning the imaging device 112-with the stored pose. The computing device 102 may provide the guidance information 175 using any combination of audio, visual, and haptic alerts.

In some aspects, the computing device 102 may provide the guidance information 175 (and information included therein) in relation to any of coordinate system tracked by the navigation system 118. For example, computing device 102 may provide the guidance information 175 in relation to any of the coordinate system of the multi-dimensional space 155-a, the coordinate system of the multi-dimensional space 155-b, the coordinate system of the environment 140, the coordinate system of the subject 141, the coordinate system of the imaging device 112, and/or the coordinate system of the robot 114 or the robotic arm 116.

The computing device 102 may adjust any combination of settings associated with the imaging device 112-b based on the guidance information 175. For example, the computing device 102 may adjust any settings of the imaging device 112-b according to stored pose information (e.g., pose information 156-a, pose information 157-a, and/or pose information 158-a). In an example, once the imaging device 112-b is aligned with the stored pose information, the computing device 102 may tune the settings of the imaging device 112-b accordingly. Example settings of the imaging device 112-b include field of view of the imaging device 112-b, image resolution, imaging duration, depth/width of signal penetration, signal transmission strength (e.g., ultrasonic energy level), etc.

In an example, once the computing device 102 determines that the imaging device 112-b is aligned with the stored pose information, the computing device 102 may deliver therapy to the subject 141 using the imaging device 112-b. In some aspects, delivering the therapy may include transmitting therapeutic ultrasound signals toward a target region. The target region may be, for example, an area corresponding to the anatomical element 142 (or portion thereof) and/or an area corresponding to the marker 143.

Aspects of the present disclosure support autonomously and/or semi-autonomously implementing the surgical plan 160 (or tasks 170 thereof) via the robot 114.

FIGS. 2A and 2B illustrates aspects of an ultrasound fan associated with an imaging device 112-b of FIG. 1B in accordance with aspects of the present disclosure. The terms “ultrasound fan” and “ultrasound fan beam” may be used interchangeably herein.

Referring to FIG. 2A, the imaging device 112-b may transmit ultrasound signals. The imaging device 112-b may capture ultrasound images based on response signals produced by the ultrasound signals. An ultrasound fan 200 formed by the ultrasound signals and response signals may correspond to the multi-dimensional space 155-c described with reference to FIG. 1B.

Features of FIG. 2A may be described in conjunction with a coordinate system 202-b. The coordinate system 202-b may be associated with any of the imaging device 112-b, multi-dimensional space 155-b, multi-dimensional space 155-c, and environment 140. The coordinate system 202-b includes two dimensions including an X2-axis and a Y2-axis. The coordinate system 202 may be used to define the X2Y2 plane). These planes may be disposed orthogonal, or at 90 degrees, to one another. In some examples, reference may be made to dimensions, angles, directions, relative positions, and/or movements associated with one or more components of the system 100 with respect to the coordinate system 202.

The ultrasound fan 200 may be segmented according to one or more lines 205 (e.g., line 205-a, line 205-b, etc.) as shown in examples 210 through 213. Example 211 illustrates an example of bisectional segmentation (also referred to herein as “bisecting”) of the ultrasound fan 200. A result of segmenting the ultrasound fan 200 may include example aspects of the multi-dimensional space 155-b of FIG. 1B. Aspects of the present disclosure support segmenting the ultrasound fan 200 in any direction.

FIG. 2B illustrates example views 220 through 240 that may be generated and displayed by a system 100 described herein. The system 100 may support displaying any of the example views 220 through 240 via separate or combined windows of a user interface 110 of FIG. 1.

Features of FIG. 2B may be described in conjunction with a coordinate system 202-a. The coordinate system 202-a may be associated with any of the imaging device 112-a, multi-dimensional space 155-a, and environment 140. The coordinate system 202-a, as shown in FIG. 2B, includes three dimensions including an X1-axis, a Y1-axis, and a Z1-axis. The coordinate system 202-a may be used to define various planes (e.g., the X1Y1 plane, the X1Z1 plane, and the Y1Z1 plane). These planes may be disposed orthogonal, or at 90 degrees, to one another. In some examples, reference may be made to dimensions, angles, directions, relative positions, and/or movements associated with one or more components of the system 100 with respect to the coordinate system 202-a.

Example view 220 includes a 3D model 225 of a portion (e.g., cranium) of a subject 141. The system 100 may support generating the example view 220 based on an exam (e.g., an MRI scan, a CT scan, a fluoroscopy scan, etc.) described herein. The system 100 may support generating an ultrasound image 235 using an ultrasound device (e.g., imaging device 112-b of FIG. 1B) and overlaying the ultrasound image 235 on the 3D model 225.

Example view 230 includes the ultrasound image 235. Example view 240 includes a 2D representation 245 of the 3D model 225. The 2D representation 245 may be referred to as a 2D slice of the 3D model 225. The system 100 may support overlaying the ultrasound image 235 on the 2D representation 245.

As illustrated in FIG. 2B, aspects of the present disclosure support navigation techniques in which the ultrasound image 235, generated using the imaging device 112-b in association with an ultrasound space, may be overlaid and oriented with respect to a coordinate system 202-a of a different multidimensional space (e.g., an MM space, a CT space, a fluoroscopy space, etc.). For example, the ultrasound image 235 may be positioned and oriented in accordance with the coordinate system 202-a, based on a mapping of pose information (e.g., coordinates, a trajectory, etc.) of the imaging device 112-b to the coordinate system 202-b. Accordingly, for example, the system 100 may support implementations in which a user may view the 3D model 225 and/or 2D representation 245 and visually identify the ultrasound trajectory associated with the ultrasound image 235 and imaging device 112-b.

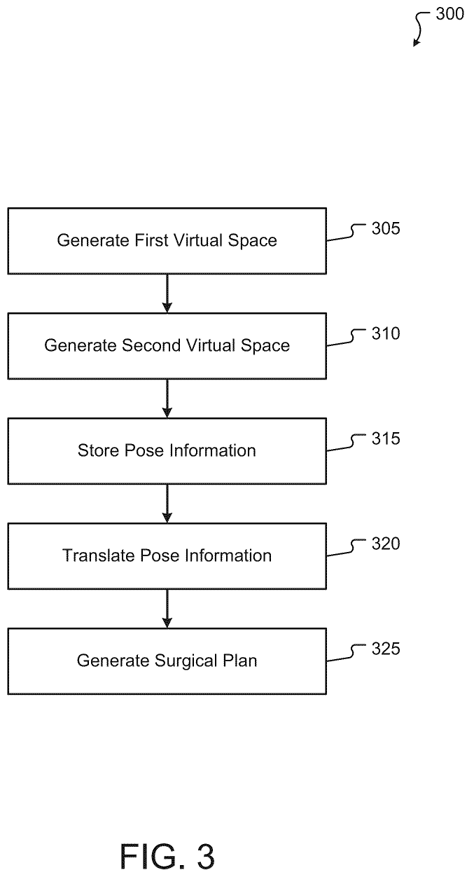

FIG. 3 illustrates an example of a process flow 300 in accordance with aspects of the present disclosure. In some examples, process flow 300 may implement aspects of a computing device 102, an imaging device 112, a robot 114, and a navigation system 118 described with reference to FIGS. 1A, 1B, and 2.

In the following description of the process flow 300, the operations may be performed in a different order than the order shown, or the operations may be performed in different orders or at different times. Certain operations may also be left out of the process flow 300, or other operations may be added to the process flow 300.

It is to be understood that any of the operations of process flow 300 may be performed by any device (e.g., a computing device 102, an imaging device 112, a robot 114, navigation system 118, etc.).

At 305, the process flow 300 may include generating a first virtual space corresponding to a first multi-dimensional image set.

In some aspects, the first multi-dimensional image set may include one or more multi-dimensional magnetic resonance imaging (Mill) images, one or more multi-dimensional computed tomography (CT) images, or one or more multi-dimensional fluoroscopic images.

In some aspects, the first multi-dimensional image set may include one or more preoperative images, one or more first intraoperative images, or both.

At 310, the process flow 300 may include generating a second virtual space. In some aspects, the second virtual space may correspond to a second multi-dimensional image set including one or more ultrasound images.

In some aspects, generating the second virtual space may include segmenting a third virtual space corresponding to the second multi-dimensional image set. In an example, the second virtual space may include a two-dimensional virtual space or a three-dimensional virtual space. In an example, the third virtual space may include a three-dimensional virtual space.

In some aspects, the second multi-dimensional image set may include one or more second preoperative images, one or more second intraoperative images, or both.

In some aspects, the process flow 300 may include: transmitting one or more ultrasound signals in a physical space corresponding to the second virtual space; and capturing the one or more ultrasound images based on the one or more ultrasound signals.

At 315, the process flow 300 may include storing pose information of one or more markers in association with the second virtual space.

In some aspects, the one or more markers correspond to one or more anatomical elements included in the one or more ultrasound images.

In some aspects, storing the pose information of the one or more markers is in response to a trigger condition.

At 320, the process flow 300 may include translating the pose information of the one or more markers to the first virtual space.

At 325, the process flow 300 may include generating an image-based surgical plan in association with the first virtual space based on the pose information of the one or more markers in association with the second virtual space. In some aspects, generating the image-based surgical plan is based on translating the pose information to the first virtual space.

In some aspects, generating the image-based surgical plan may include mapping one or more parameters of a surgical task included in the image-based surgical plan to the pose information of the one or more markers.

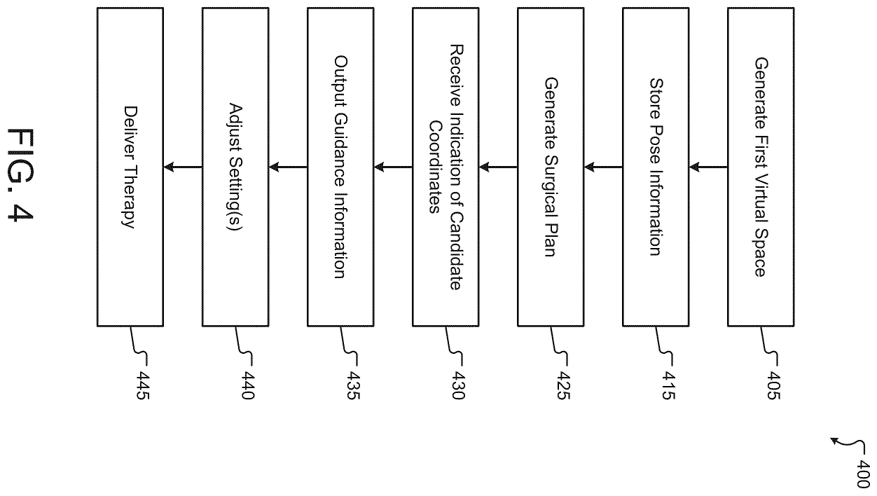

FIG. 4 illustrates an example of a process flow 400 in accordance with aspects of the present disclosure. In some examples, process flow 400 may implement aspects of a computing device 102, an imaging device 112, a robot 114, and a navigation system 118 described with reference to FIGS. 1A, 1B, and 2.

In the following description of the process flow 400, the operations may be performed in a different order than the order shown, or the operations may be performed in different orders or at different times. Certain operations may also be left out of the process flow 400, or other operations may be added to the process flow 400.

It is to be understood that any of the operations of process flow 400 may be performed by any device (e.g., a computing device 102, an imaging device 112, a robot 114, navigation system 118, etc.).

At 405, the process flow 400 may include generating a first virtual space corresponding to a first multi-dimensional image set.

At 415, the process flow 400 may include storing pose information of one or more markers in association with a second virtual space. In some aspects, the second virtual space may correspond to a second multi-dimensional image set including one or more ultrasound images.

At 425, the process flow 400 may include generating an image-based surgical plan in association with the first virtual space based on the pose information of the one or more markers in association with the second virtual space.

At 430, the process flow 400 may include receiving an indication of candidate coordinates associated with the image-based surgical plan and the first virtual space.

At 435, the process flow 400 may include outputting, in response to receiving the indication of the candidate coordinates, guidance information associated with at least the image-based surgical plan.

In some aspects, the guidance information may include the pose information of the one or more markers in association with the second virtual space. In some aspects, the pose information of the one or more markers corresponds to the candidate coordinates in the first virtual space.

In some aspects, the guidance information may include an indication of a target point in the second virtual space. In some aspects, the target point is associated with the one or more markers.

In some aspects, the guidance information may include an indication of at least one of: a target pose of an image sensor device with respect to a target point in the second virtual space; a target trajectory of the image sensor device with respect to the target point in the second virtual space; and a hind point of the target trajectory.

In some aspects, the guidance information may include an indication of at least one of: a target pose of an image sensor device with respect to a target point in a physical space corresponding to the second virtual space; a target trajectory of the image sensor device with respect to the target point in the physical space; and a hind point of the target trajectory.

In some aspects, the guidance information may include alignment information associated with current pose information of an image sensor device and stored pose information of the image sensor device; and the stored pose information of the image sensor device correlates to the candidate coordinates associated with the image-based surgical plan and the first virtual space.

At 440, the process flow 400 may include adjusting one or more settings associated with an image sensor device based on the guidance information.

At 445, the process flow 400 may include delivering therapy to a subject based on at least one of the one or more markers and the image-based surgical plan. In some aspects, delivering the therapy may include transmitting one or more therapeutic ultrasound signals toward a region associated with the one or more markers. In some aspects, the process flow 400 may include delivering diagnostics data associated with the subject based on at least one of the one or more markers and the image-based surgical plan. For example, the diagnostics data may be associated with an anatomical element of the subject located at the one or more markers. In some aspects, the process flow 400 may include generating and delivering the diagnostics data in response to delivering the therapy to the subject (e.g., to evaluate the impact of delivering the therapy).

The process flows 300 and 400 (and/or one or more operations thereof) may be carried out or otherwise performed, for example, by at least one processor. The at least one processor may be the same as or similar to the processor(s) 104 of the computing device 102 described above. The at least one processor may be part of a robot (such as a robot 114) or part of a navigation system (such as a navigation system 118). A processor other than any processor described herein may also be used to execute the process flows 300 and 400. The at least one processor may perform operations of the process flow 300 and 400 by executing elements stored in a memory such as the memory 106. The elements stored in memory and executed by the processor may cause the processor to execute one or more operations of a function as shown in the process flows 300 and 400. One or more portions of the process flows 300 and 400 may be performed by the processor 104 executing any of the contents of memory.

As noted above, the present disclosure encompasses methods with fewer than all of the steps identified in FIGS. 3 and 4 (and the corresponding description of the process flows 300 and 400), as well as methods that include additional steps beyond those identified in FIGS. 3 and 4 (and the corresponding description of the process flows 300 and 400). The present disclosure also encompasses methods that include one or more steps from one method described herein, and one or more steps from another method described herein. Any correlation described herein may be or include a registration or any other correlation.

The foregoing is not intended to limit the disclosure to the form or forms disclosed herein. In the foregoing Detailed Description, for example, various features of the disclosure are grouped together in one or more aspects, implementations, and/or configurations for the purpose of streamlining the disclosure. The features of the aspects, implementations, and/or configurations of the disclosure may be combined in alternate aspects, implementations, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, implementation, and/or configuration. Thus, the following claims are hereby incorporated into this Detailed Description, with each claim standing on its own as a separate preferred implementation of the disclosure.

Moreover, though the foregoing has included description of one or more aspects, implementations, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, implementations, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

Example aspects of the present disclosure include:

A system including: a processor; and a memory storing instructions thereon that, when executed by the processor, cause the processor to: generate a first virtual space corresponding to a first multi-dimensional image set; store pose information of one or more markers in association with a second virtual space, wherein the second virtual space corresponds to a second multi-dimensional image set including one or more ultrasound images; and generate an image-based surgical plan in association with the first virtual space based on the pose information of the one or more markers in association with the second virtual space.

Any of the aspects herein, wherein the instructions are further executable by the processor to: translate the pose information of the one or more markers to the first virtual space, wherein generating the image-based surgical plan is based on translating the pose information to the first virtual space.

Any of the aspects herein, further including generating the second virtual space, wherein: generating the second virtual space includes segmenting a third virtual space corresponding to the second multi-dimensional image set; the second virtual space includes a two-dimensional virtual space or a three-dimensional virtual space; and the third virtual space includes a three-dimensional virtual space.

Any of the aspects herein, wherein the instructions are further executable by the processor to: receive an indication of candidate coordinates associated with the image-based surgical plan and the first virtual space; and output, in response to receiving the indication of the candidate coordinates, guidance information associated with at least the image-based surgical plan.

Any of the aspects herein, wherein: the guidance information includes the pose information of the one or more markers in association with the second virtual space; and the pose information of the one or more markers corresponds to the candidate coordinates in the first virtual space.

Any of the aspects herein, wherein the guidance information includes an indication of a target point in the second virtual space, wherein the target point is associated with the one or more markers.

Any of the aspects herein, wherein the guidance information includes an indication of at least one of: a target pose of an image sensor device with respect to a target point in the second virtual space; a target trajectory of the image sensor device with respect to the target point in the second virtual space; and a hind point of the target trajectory.

Any of the aspects herein, wherein the guidance information includes an indication of at least one of: a target pose of an image sensor device with respect to a target point in a physical space corresponding to the second virtual space; a target trajectory of the image sensor device with respect to the target point in the physical space; and a hind point of the target trajectory.

Any of the aspects herein, wherein: the guidance information includes alignment information associated with current pose information of an image sensor device and stored pose information of the image sensor device; and the stored pose information of the image sensor device correlates to the candidate coordinates associated with the image-based surgical plan and the first virtual space.

Any of the aspects herein, wherein the instructions are further executable by the processor to adjust one or more settings associated with an image sensor device based on the guidance information.

Any of the aspects herein, wherein the instructions are further executable by the processor to at least one of: deliver therapy to a subject based on at least one of the one or more markers and the image-based surgical plan, wherein delivering the therapy includes transmitting one or more therapeutic ultrasound signals toward a region associated with the one or more markers; and deliver diagnostics data associated with the subject based on at least one of the one or more markers and the image-based surgical plan.

Any of the aspects herein, wherein the one or more markers correspond to one or more anatomical elements included in the one or more ultrasound images.

Any of the aspects herein, wherein generating the image-based surgical plan includes mapping one or more parameters of a surgical task included in the image-based surgical plan to the pose information of the one or more markers.

Any of the aspects herein, wherein storing the pose information of the one or more markers is in response to a trigger condition.

Any of the aspects herein, wherein the instructions are further executable by the processor to: transmit one or more ultrasound signals in a physical space corresponding to the second virtual space; and capture the one or more ultrasound images based on the one or more ultrasound signals.

Any of the aspects herein, wherein: wherein the first multi-dimensional image set includes one or more magnetic resonance imaging (MM) images, one or more computed tomography (CT) images, or one or more multi-dimensional fluoroscopic images.

Any of the aspects herein, wherein: the first multi-dimensional image set includes one or more preoperative images, one or more first intraoperative images, or both; and the second multi-dimensional image set includes one or more second preoperative images, one or more second intraoperative images, or both.

A system including: an interface to receive one or more imaging signals; a processor; and a memory storing data thereon that, when processed by the processor, cause the processor to: generate a first virtual space corresponding to a first multi-dimensional image set, wherein the first multi-dimensional image set includes one or more images generated based on first imaging signals; store pose information of one or more markers in association with a second virtual space, wherein the second virtual space corresponds to a second multi-dimensional image set including one or more ultrasound images generated based on second imaging signals; and generate an image-based surgical plan in association with the first virtual space based on the pose information of the one or more markers.

Any of the aspects herein, wherein the instructions are further executable by the processor to: translate the pose information of the one or more markers to the first virtual space, wherein generating the image-based surgical plan is based on translating the pose information to the first virtual space.

A method including: generating a first virtual space corresponding to a first multi-dimensional image set, wherein the first multi-dimensional image set includes one or more images generated based on receiving first imaging signals; storing pose information of one or more markers in association with a second virtual space, wherein the second virtual space corresponds to a second multi-dimensional image set including one or more ultrasound images generated based on receiving second imaging signals; and generating an image-based surgical plan in association with the first virtual space based on the pose information of the one or more markers.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/implementations in combination with any one or more other aspects/features/implementations.

Use of any one or more of the aspects or features as disclosed herein.

It is to be appreciated that any feature described herein can be claimed in combination with any other feature(s) as described herein, regardless of whether the features come from the same described implementation.

The phrases “at least one,” “one or more,” “or,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” “A, B, and/or C,” and “A, B, or C” means A alone, B alone, C alone, A and B together, A and C together, B and C together, or A, B and C together.

The term “a” or “an” entity refers to one or more of that entity. As such, the terms “a” (or “an”), “one or more,” and “at least one” can be used interchangeably herein. It is also to be noted that the terms “comprising,” “including,” and “having” can be used interchangeably.

The term “automatic” and variations thereof, as used herein, refers to any process or operation, which is typically continuous or semi-continuous, done without material human input when the process or operation is performed. However, a process or operation can be automatic, even though performance of the process or operation uses material or immaterial human input, if the input is received before performance of the process or operation. Human input is deemed to be material if such input influences how the process or operation will be performed. Human input that consents to the performance of the process or operation is not deemed to be “material.”

Aspects of the present disclosure may take the form of an implementation that is entirely hardware, an implementation that is entirely software (including firmware, resident software, micro-code, etc.) or an implementation combining software and hardware aspects that may all generally be referred to herein as a “circuit,” “module,” or “system.” Any combination of one or more computer-readable medium(s) may be utilized. The computer-readable medium may be a computer-readable signal medium or a computer-readable storage medium.

A computer-readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer-readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer-readable storage medium may be any tangible medium that can contain or store a program for use by or in connection with an instruction execution system, apparatus, or device.

A computer-readable signal medium may include a propagated data signal with computer-readable program code embodied therein, for example, in baseband or as part of a carrier wave. Such a propagated signal may take any of a variety of forms, including, but not limited to, electro-magnetic, optical, or any suitable combination thereof. A computer-readable signal medium may be any computer-readable medium that is not a computer-readable storage medium and that can communicate, propagate, or transport a program for use by or in connection with an instruction execution system, apparatus, or device. Program code embodied on a computer-readable medium may be transmitted using any appropriate medium, including, but not limited to, wireless, wireline, optical fiber cable, RF, etc., or any suitable combination of the foregoing.

The terms “determine,” “calculate,” “compute,” and variations thereof, as used herein, are used interchangeably and include any type of methodology, process, mathematical operation or technique.

What is claimed is:

1. A system comprising:
a processor; and
a memory storing instructions thereon that, when executed by the processor, cause the processor to:
generate a first virtual space corresponding to a first multi-dimensional image set;
store pose information of one or more markers in association with a second virtual space, wherein the second virtual space corresponds to a second multi-dimensional image set comprising one or more ultrasound images; and
generate an image-based surgical plan in association with the first virtual space based on the pose information of the one or more markers in association with the second virtual space.

2. The system of claim 1, wherein the instructions are further executable by the processor to:
translate the pose information of the one or more markers to the first virtual space,
wherein generating the image-based surgical plan is based on translating the pose information to the first virtual space.

3. The system of claim 1, further comprising generating the second virtual space, wherein:
generating the second virtual space comprises segmenting a third virtual space corresponding to the second multi-dimensional image set;
the second virtual space comprises a two-dimensional virtual space or a three-dimensional virtual space; and
the third virtual space comprises a three-dimensional virtual space.

4. The system of claim 1, wherein the instructions are further executable by the processor to:
receive an indication of candidate coordinates associated with the image-based surgical plan and the first virtual space; and
output, in response to receiving the indication of the candidate coordinates, guidance information associated with at least the image-based surgical plan.

5. The system of claim 4, wherein:
the guidance information comprises the pose information of the one or more markers in association with the second virtual space; and
the pose information of the one or more markers corresponds to the candidate coordinates in the first virtual space.

6. The system of claim 4, wherein the guidance information comprises an indication of a target point in the second virtual space, wherein the target point is associated with the one or more markers.

7. The system of claim 4, wherein the guidance information comprises an indication of at least one of:
a target pose of an image sensor device with respect to a target point in the second virtual space;
a target trajectory of the image sensor device with respect to the target point in the second virtual space; and
a hind point of the target trajectory.

8. The system of claim 4, wherein the guidance information comprises an indication of at least one of:
a target pose of an image sensor device with respect to a target point in a physical space corresponding to the second virtual space;
a target trajectory of the image sensor device with respect to the target point in the physical space; and
a hind point of the target trajectory.

9. The system of claim 4, wherein:
the guidance information comprises alignment information associated with current pose information of an image sensor device and stored pose information of the image sensor device; and
the stored pose information of the image sensor device correlates to the candidate coordinates associated with the image-based surgical plan and the first virtual space.

10. The system of claim 4, wherein the instructions are further executable by the processor to adjust one or more settings associated with an image sensor device based on the guidance information.

11. The system of claim 1, wherein the instructions are further executable by the processor to at least one of:
deliver therapy to a subject based on at least one of the one or more markers and the image-based surgical plan, wherein delivering the therapy comprises transmitting one or more therapeutic ultrasound signals toward a region associated with the one or more markers; and
deliver diagnostics data associated with the subject based on at least one of the one or more markers and the image-based surgical plan.

12. The system of claim 1, wherein the one or more markers correspond to one or more anatomical elements comprised in the one or more ultrasound images.

13. The system of claim 1, wherein generating the image-based surgical plan comprises mapping one or more parameters of a surgical task included in the image-based surgical plan to the pose information of the one or more markers.

14. The system of claim 1, wherein storing the pose information of the one or more markers is in response to a trigger condition.

15. The system of claim 1, wherein the instructions are further executable by the processor to:
transmit one or more ultrasound signals in a physical space corresponding to the second virtual space; and
capture the one or more ultrasound images based on the one or more ultrasound signals.

16. The system of claim 1, wherein:
wherein the first multi-dimensional image set comprises one or more magnetic resonance imaging (MRI) images, one or more computed tomography (CT) images, or one or more multi-dimensional fluoroscopic images.

17. The system of claim 1, wherein:
the first multi-dimensional image set comprises one or more preoperative images, one or more first intraoperative images, or both; and
the second multi-dimensional image set comprises one or more second preoperative images, one or more second intraoperative images, or both.

18. A system comprising:
an interface to receive one or more imaging signals;
a processor; and
a memory storing data thereon that, when processed by the processor, cause the processor to:
generate a first virtual space corresponding to a first multi-dimensional image set, wherein the first multi-dimensional image set comprises one or more images generated based on first imaging signals;
store pose information of one or more markers in association with a second virtual space, wherein the second virtual space corresponds to a second multi-dimensional image set comprising one or more ultrasound images generated based on second imaging signals; and
generate an image-based surgical plan in association with the first virtual space based on the pose information of the one or more markers.

19. The system of claim 18, wherein the instructions are further executable by the processor to:
translate the pose information of the one or more markers to the first virtual space,
wherein generating the image-based surgical plan is based on translating the pose information to the first virtual space.

20. A method comprising:
generating a first virtual space corresponding to a first multi-dimensional image set, wherein the first multi-dimensional image set comprises one or more images generated based on receiving first imaging signals;
storing pose information of one or more markers in association with a second virtual space, wherein the second virtual space corresponds to a second multi-dimensional image set comprising one or more ultrasound images generated based on receiving second imaging signals; and
generating an image-based surgical plan in association with the first virtual space based on the pose information of the one or more markers.
